(19) United States
(12) Patent Application Publication
(10) Pub. No.: US 2014/0348850 A1
(43) Pub. Date: Nov. 27, 2014

(54) METHOD FOR REDUCING BLOOD PRESSURE USING INHIBITORS OF PLASMA KALLIKREIN

(71) Applicant: JOSLIN DIABETES CENTER, Boston, MA (US)

(72) Inventors: Edward P. Feener, North Reading, MA (US); Allen Clermont, Stoughton, MA (US)

(21) Appl. No.: 14/460,679

(22) Filed: Aug. 15, 2014

Related U.S. Application Data
(63) Continuation of application No. 13/132,152, filed on Sep. 16, 2011, now abandoned.
(60) Provisional application No. 61/200,600, filed on Dec. 2, 2008.

(51) Int. Cl. C07K 16/40; C07D 401/06; A61K 31/415; C07D 231/14; A61K 31/341; C07D 307/68; A61K 31/4192; C07D 249/04; A61K 31/381; C07D 333/38; A61K 31/426; C07D 277/56; A61K 31/4436; C07D 409/04; A61K 31/40; C07D 207/34; A61K 39/395; A61K 38/00; A61K 38/02; A61K 45/06; A61K 31/4439

(52) U.S. Cl. CPC C07K 16/40; A61K 31/4439; C07D 401/06; A61K 31/415; C07D 231/14; A61K 31/341; C07D 307/68; A61K 31/4192; C07D 249/04; A61K 31/381; C07D 333/38; A61K 31/426; C07D 277/56; A61K 31/4436; C07D 409/04; A61K 31/40; C07D 207/34; A61K 39/3955; A61K 38/005; A61K 38/02; A61K 45/06
USPC 424/146.1; 514/343; 546/279.1; 514/406; 548/374.1; 514/471; 549/487; 514/359; 548/255; 514/448; 549/72; 514/365; 548/200; 514/336; 546/280.4; 514/423; 548/537; 530/388.26; 514/15.7; 530/317

(57) ABSTRACT

The present invention relates to methods of reducing blood pressure in a subject by administering a plasma kallikrein inhibitor.

### BACKGROUND OF THE INVENTION

The invention relates to methods of reducing blood pressure using inhibitors of plasma kallikrein in subjects, for example, those suffering from hypertension.

Hypertension, or persistently elevated blood pressure (BP), is estimated to affect 72 million people in the United States alone (Rosamond et al., Circulation 115:e69-e171, 2007), increases both microvascular (retinopathy, stroke, nephropathy) and macrovascular (myocardial infarction) diseases, and can lead to organ failure. The increased risk of cardiovascular disease (CVD) conferred by elevated BP has been shown to be much higher when the hypertension co-exists with other CVD risk factors, such as diabetes or hyperlipidemia. Although traditionally hypertension is defined as systolic BP (SBP)/diastolic BP (DBP) exceeding 140/90 mm Hg, incremental increases in BP in the “normal” range has been shown to increase the risk of vascular disease (Flack et al, Am J Kid Dis 21:S31-S40, 1993). The Treatment of Mild Hypertension Study (TOMHS) reported fewer CVD events in apparently low-risk patients with hypertension when the SBP was reduced, on average, from 131 to 125 mm Hg (Neaton et al, JAMA 270:713-724, 1993). Sustained reduction of BP in patients affected with hypertension is therefore widely considered to be a key goal in reducing the risk of vascular events and CVD in the general population.

Pharmacological reduction of BP is usually achieved by prescribing one or more of the following classes of drugs: diuretics (e.g., hydrochlorothiazide), beta-adrenergic receptor blockers (e.g., atenolol), calcium channel blockers (e.g., amlodipine), angiotensin-converting enzyme (ACE) inhibitors (e.g., benazepril) and AT1 receptor blockers (ARBs) (e.g., losartan). Even so, pharmacological control of BP in diagnosed hypertensives remain suboptimal, primarily because of a reliance on monotherapy, where even high adherence (>80%) to medication leads to only a 43% incidence of targeted BP control. Multidrug therapy has been recognized as the most effective method of achieving target BP control, and it has been recommended that this should be pursued aggressively for optimal BP control (Chobanian et al, JAMA, 289:2560-72, 2003). There is thus a major unmet need for new pharmacological agents, which when combined with one or more existing drugs can lead to improved BP control.

One class of anti-hypertensive drugs, the ARBs, has received renewed attention recently. In patients with hypertension who have additional risk factors, suppression of the action of the renin-angiotensin system (RAS) with ARBs has been shown to be superior to other classes of medication, especially in affording protection against renal failure and heart failure (Brenner et al, N Engl J Med, 345:851-60, 2001). ARBs block the action of the peptide angiotensin-II (Ang-II) on AT1 receptors. This suggests that the action of Ang-II may play an especially important role in the pathological manifestations of hypertension. Ang-II is the key peptide produced by the RAS, formed from its precursor angiotensinogen, as a consequence of sequential action of two proteolytic enzymes, renin and ACE. Although Ang-II has multiple physiological effects mediated via its action on two distinct receptors subtypes (AT1 and AT2), elevation of BP in hypertension is known to be mediated via Ang-11 action on the AT1 receptor (Ito et al, Proc Natl Acad Sci USA 92:3521-3525, 1995).

In fact, a widely used rodent model of slowly developing hypertension utilizes continuous infusion of a low dose of Ang-II (10-100 ng/kg/min), achieved by a pump implanted subcutaneously or intraperitoneally (Pelaez et al, Hypertension, 42:798-801, 2003). Higher doses (>150 ng/kg/min) lead to elevated BP occurring more readily within a few days. These models have proven very useful in studying both the physiological activities of Ang-II, as well as evaluating the action of both established and novel drugs in antagonizing or potentiating Ang-II action.

### SUMMARY OF THE INVENTION

We have discovered that plasma kallikrein inhibitors, as exemplified by the compound ASP-440 (1-benzyl-1H-pyrazole-4-carboxylic acid 4-carbamimidoyl-benzylamide), are useful in decreasing BP induced by infusion of Ang-II.

The present invention therefore features a method for reducing blood pressure in a subject (e.g., a human) in need thereof, by administering to the subject an effective amount of an inhibitor of plasma kallikrein. In certain embodiments, the systolic blood pressure (SBP) of the subject is greater than 120 (e.g., 122, 125, 130, 135, 139, 140, or 150) mm Hg, the diastolic blood pressure of the subject is greater than 80 (e.g., 81, 83, 85, 87, 89, 90, 95, 100, or 110) mm Hg, or a combination thereof. The subject may suffer from primary hypertension or from secondary hypertension (e.g., caused by any disease or condition known in the art such as those described herein). The subject may have or be at increased risk of developing angioedema (e.g., angioedema resulting from the use of angiotensin-converting enzyme (ACE) inhibitors). In certain embodiments, the subject is prehypertensive. The inhibitor may be a selective plasma kallikrein inhibitor. The inhibitor may be a naturally occurring compound (e.g., Cl-Inhibitor or any naturally occurring compound described herein). In other embodiments, the inhibitor is a non-naturally occurring compound (e.g., a compound produced recombinantly such as ecallantide (DX-88) or a bicyclic peptide such as PK1, PK2, PK3, P1(4, PK5, PK6, PK7, PK8, PK9, PK10, PK11, PK12, PK13, PKI 4, PK15, PK16, PK17, PK18, PK19, PK20, PIC21, PK22, and PK23). The plasma kallikrein inhibitor may be chemically modified to increase oral uptake or bioavailability, for example, as a prodrug.

In one aspect of the invention, an effective amount of a compound having the formula I or formula II is administered. In one embodiment, the compound has the formula (I):

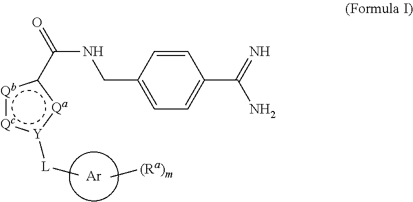

where Ar is a bond or an aromatic ring selected from the group consisting of benzene, pyridine and pyrimidine. When Ar is a bond, m is 1. When Ar is an aromatic ring, m is an integer from 0-5. In one embodiment, Ar is benzene or pyridine. In another embodiment, Ar is a bond.

The subscript m is an integer from 0 to 5. In one embodiment, m is 0. Each R^{a} is independently selected from the group consisting of cycloalkyl, haloalkyl, halogen, —OH, —OSi(R¹)₃, —OC(O)O—R¹, —OC(O)R′, —OC(O)NHR¹, —OC(O)N(R¹)₂, —SH, —SR¹, —S(O)R¹, —S(O)₂R¹, —SO₂NH₂, —S(O)₂NHR¹, —S(O)₂N(R¹)₂, —NHS(O)₂R¹, —NR¹S(O)₂R¹, —C(O)NH₂, —C(O)NHR¹, —C(O)N(R¹)₂, —C(O)R¹, —C(O)H, —NHC(O)R′, —NR′C(O)R¹, —NHC(O)NH₂, —NR′C(O)NH₂, —NR¹C(O)NHR¹, —NHC(O)NHR¹, —NR¹C(O)N(R¹)₂, —NHC(O)N(R¹)₂, —CO₂H, —CO₂R¹, —NHCO₂R¹, —NR′CO₂R¹, —R¹, —CN, —NO₂, —NH₂, —NHR¹, —N(R¹)₂, —NR¹S(O)NH₂, —NR¹S(O)₂NHR¹, —NH₂C(═NR¹)NH₂, —N═C(NH₂)NH₂, —C(═NR¹)NH₂, —NH—OH, —NR¹—OH, —NR¹—OR¹, —N═C═O, —Si(R¹)₃, —NH—NHR¹, —NHC(O)NHNH₂, NO, —N═C≡NR¹ and —S—CN, wherein each R¹ is independently alkyl (e.g., C₁-C₈ alkyl), aryl (e.g., C₆-C₁₂ aryl), or arylalkyl (e.g., C₇-C₁₄ arylalkyl). In one embodiment, R′ is C₁-C₈ alkyl. In another embodiment, R′ is unsubstituted aryl, such as phenyl or pyridyl, or a substituted aryl, such as a substituted phenyl or a substituted pyridyl.

In one embodiment, each R^{a} is independently selected from the group consisting of C₁-C₈ alkyl, C₁-C₈ alkoxy, aryl, aryl(C₁-C₈ alkyl), halogen, —NH₂, —NH(C₁-C₈ alkyl), —N(C₁-C₈ alkyl)₂, —CN, —C(═O)(C₁-C₈ alkyl), —(C═O)NH₂, —(C═O)NH(C₁-C₈ alkyl), —C(═O)N(C₁-C₈ alkyl)₂, —OH, —COOH, —COO(C₁-C₈ alkyl), —OCH(C₁-C₈ alkyl), —O(C═O)O(C₁-C₈ alkyl)-NO₂, —SH, —S(C₁-C₈ alkyl), —NH(C═O)(C₁-C₈ alkyl), —NH(C═O)O(C₁-C₈ alkyl), —O(C═O)NH(C₁-C₈ alkyl), —SO₂(C₁-C₈ alkyl), —NHSO₂(C₁-C₈ alkyl) and —SO₂NH(C₁-C₈ alkyl). In another embodiment, each le is independently selected from the group consisting of C₁-C₈ alkyl, C₁-C₈ alkoxy, phenyl, phenyl (C₁-C₈ alkyl), halogen, —CN, —NH₂, —NH(C₁-C₈ alkyl), —N(C₁-C₈ alkyl)₂, —(C═O)CH₃, —(C═O)NH₂, —OH, —COOH, —COO(C₁-C₈ alkyl), —OCH(C₁-C₈ alkyl), —O(C═O)O(C₁-C₈ alkyl), —NO₂, —SH, —S(C₁-C₈ alkyl), and —NH(C═O)(C₁-C₈ alkyl). In yet another embodiment, each R^{a} is independently selected from the group consisting of C₁-C₈ alkyl, Cᵣ—C₈ alkoxy, phenyl, phenyl (C₁-C₈ alkyl), phenoxy, aryloxy, halogen, —CN, —NH₂, —NH-aryl, —(C═O)CH₃, —(C═O)NH₂, —OH, —COOH, —COO(C₁-C₈ alkyl), —OCO(C₁-C₈ alkyl), —COO-aryl, —OC(O)-aryl, —O(C═O)O(C₁-C₈ alkyl)-NO₂, —SH, —S(C₁-C₈ alkyl), —NH(C═O)(C₁-C₈ alkyl), and the like. For example, le is halogen, such as Cl, Br, or I.

L is a linking group selected from the group consisting of a bond, CH₂ and SO₂.

Q^{a}, Q^{b}, and Q^{c} are each members independently selected from the group consisting of N, S, O and C(R^{q}) wherein each R^{q} is independently selected from the group consisting of H, C₁₋₈ alkyl, halogen and phenyl, and the ring having Q^{a}, Q^{b}, Q^{c}, and Y as ring vertices is a five-membered ring having two double bonds.

In a first group of embodiments, Q^{a} is N and Q^{b} and Q^{c} are each selected from N, O, and C(R^{q}). In certain instances, Q^{a} is N and Q^{c} and Q^{b} are each independently selected from N and C(R^{q}). In certain other instances, Q^{a} is N and Q^{c} and Q^{b} are each selected from C(R^{q}) and O. In yet certain other instances, Q^{a} is N, Q^{c} is a member selected from N and O, and Q^{b} is the other member selected from N and O.

In a second group of embodiments, Q^{a} is O and Q^{b} and Q^{c} are each selected from N, O, and C(R^{q}). In certain instances, Q^{a} is O and Q^{b} and Q^{c} are each independently selected from N and C(R^{q}).

In a third group of embodiments, Q⁸ is C(R^{q}) and Q^{b} and Q^{c} are each selected from N, O and C(R^{q}). In certain instances, Q^{a} is C(R^{q}) and Q^{b} and Q^{c} are each independently selected from N and O. In certain other instances, Q^{a} is C(R^{q}) and Q^{b} and Q^{c} are each independently selected from N and C(R^{q}). In yet certain other instances, Q^{a} is C(R^{q}) and Q^{b} and Q^{c} are each independently selected from O and C(R^{q}). In one occurrence, Q^{a} is C(R^{q}), Q^{b} is O and Q^{c} is (Cltq).

Y is a member selected from the group consisting of C and N. In one embodiment, Y is C, Q^{a} is S and Ar is selected from phenyl and pyridyl. In another embodiment, Y is N, Q^{a}, Q^{b} and Q^{c} are each independently C(R^{q}), wherein R^{q} is H or C₁₋₈alkyl. In one instance, Y is N, Q^{a} and Q^{c} are C(R^{q}) and Q^{b} is CH. In a preferred embodiment, Y is N.

In one embodiment, L is a bond, Y is N. In another embodiment, L is a bond, Y is N, and Ar is a benzene ring. In yet another embodiment, L is CH₂ and Y is N. In still another embodiment, L is a bond and Y is C. In a further embodiment, L is SO₂ and Y is N.

In a preferred embodiment, Q^{a}, Q^{b}, and Q^{c} are each independently CR^{q}. In another preferred embodiment, L is a bond or CH₂. In still another preferred embodiment, Ar is benzene. In still another preferred embodiment, le is —H and C₁-C₈ alkyl.

Particular compounds of formula I are set forth in Table 1 below. Further compounds include ASP-587.

TABLE 1

| | |
|---|---|
| 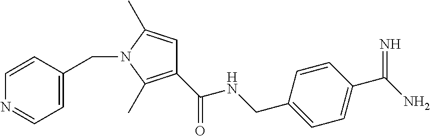 [SMILES: Cc1cc(C(=O)NCc2ccc(C(=N)N)cc2)c(C)n1Cc1ccncc1] | ASP-465 |
| 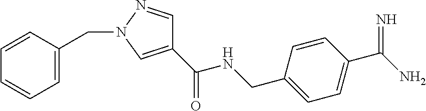 [SMILES: N=C(N)c1ccc(CNC(=O)c2cnn(Cc3ccccc3)c2)cc1] | ASP-440 |
| 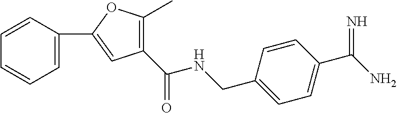 [SMILES: Cc1oc(-c2ccccc2)cc1C(=O)NCc1ccc(C(=N)N)cc1] | ASP-466 |
| 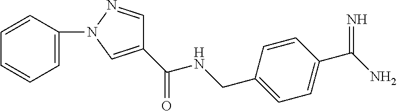 [SMILES: N=C(N)c1ccc(CNC(=O)c2cnn(-c3ccccc3)c2)cc1] | ASP-445 |
| 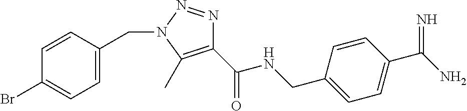 [SMILES: Cc1c(C(=O)NCc2ccc(C(=N)N)cc2)nnn1Cc1ccc(Br)cc1] | ASP-558 |
| 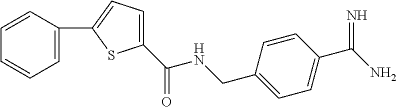 [SMILES: N=C(N)c1ccc(CNC(=O)c2ccc(-c3ccccc3)s2)cc1] | ASP-523 |
| 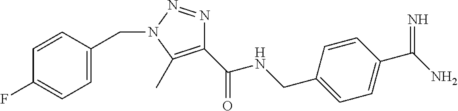 [SMILES: Cc1c(C(=O)NCc2ccc(C(=N)N)cc2)nnn1Cc1ccc(F)cc1] | ASP-559 |
| 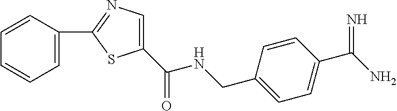 [SMILES: N=C(N)c1ccc(CNC(=O)c2cnc(-c3ccccc3)s2)cc1] | ASP-373 |
| 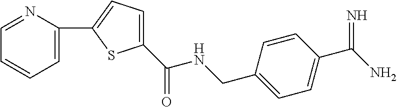 [SMILES: N=C(N)c1ccc(CNC(=O)c2ccc(-c3ccccn3)s2)cc1] | ASP-525 |
| 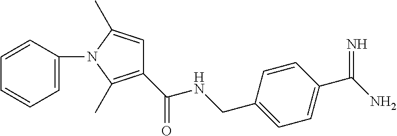 [SMILES: Cc1cc(C(=O)NCc2ccc(C(=N)N)cc2)c(C)n1-c1ccccc1] | ASP-576 |
| 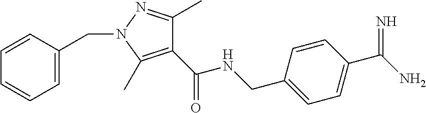 [SMILES: Cc1nn(Cc2ccccc2)c(C)c1C(=O)NCc1ccc(C(=N)N)cc1] | ASP-577 |
| 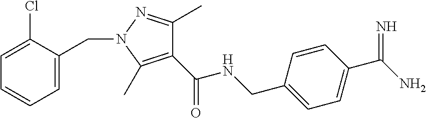 [SMILES: Cc1nn(Cc2ccccc2Cl)c(C)c1C(=O)NCc1ccc(C(=N)N)cc1] | ASP-578 |
| 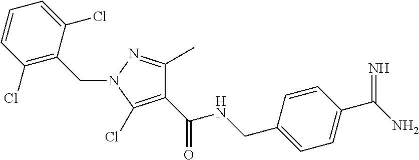 [SMILES: Cc1nn(Cc2c(Cl)cccc2Cl)c(Cl)c1C(=O)NCc1ccc(C(=N)N)cc1] | ASP-579 |
| 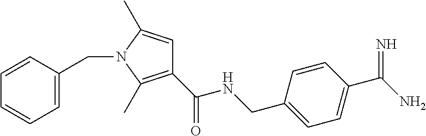 [SMILES: Cc1cc(C(=O)NCc2ccc(C(=N)N)cc2)c(C)n1Cc1ccccc1] | ASP-580 |
| 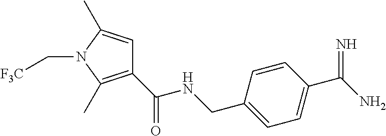 [SMILES: Cc1cc(C(=O)NCc2ccc(C(=N)N)cc2)c(C)n1CC(F)(F)F] | ASP-581 |
| 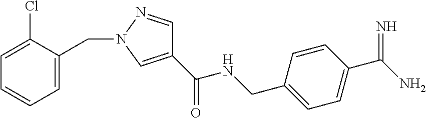 [SMILES: N=C(N)c1ccc(CNC(=O)c2cnn(Cc3ccccc3Cl)c2)cc1] | ASP-582 |
| 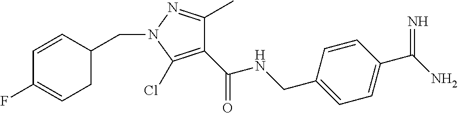 [SMILES: Cc1nn(CC2C=CC(F)=CC2)c(Cl)c1C(=O)NCc1ccc(C(=N)N)cc1] | ASP-583 |

In another embodiment, the compound of formula I has a subformula Ia:

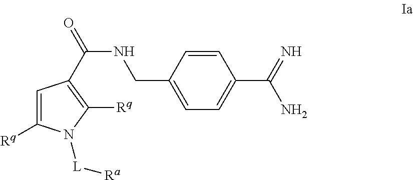

where Rq and L are as defined above. In one instance, R^{q} is independently —H or C₁₋₈ alkyl and L is a bond or —CH₂—. In another instance, R^{a} is halo-(Cₜ—C₈ alkyl). For example, R^{a} is —CF₃, CH₂CF₃.

In one embodiment, the compounds of formula I have a subformula Ib:

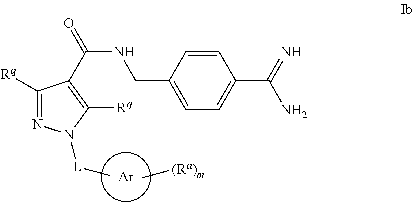

wherein Ar is an aromatic ring. In one instance, each R^{q} is independently H, C₁-C₈ alkyl, or halogen. In another instance, L is a bond or CH₂. In yet another instance, Ar is benzene. In still another instance, m is 0.

In one occurrence, each R^{q} is H, L is CH₂, Ar is benzene, and m is 0. In another occurrence, each R^{q} is H, L is a bond, Ar is benzene, and m is 0.

In another aspect, the present invention includes the use of a compound having the formula II:

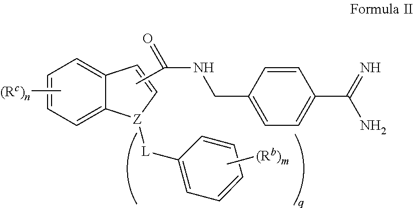

The subscript m is an integer of from 0 to 5. The subscript n is an integer of from 0 to 4. The subscript q is an integer of from 0 to 1. In one embodiment, the subscript m is 0. In another embodiment, the subscript n is an integer from 0 to 2. In yet another embodiment, the subscript q is 0. In still another embodiment, the subscript q is 1.

L is a linking group selected from the group consisting of a bond, CH₂ and SO₂. In one embodiment, L is CH₂ or SO₂.

Each of R^{b} and R^{c} is independently selected from the group consisting of cycloalkyl, haloalkyl, halogen, —OH, —OR², —OSi(R²)₃, —OC(O)O—R², —OC(O)R², —OC(O)NHR², —OC(O)N(R²)₂, —SH, —SR², —S(O)R², —S(O)₂R², —SO₂NH₂, —S(O)₂NHR², —S(O)₂N(R²)₂, —NHS(O)₂R², —NR²S(O)₂R², —C(O)NH₂, —C(O)NHR², —C(O)N(R²)₂, —C(O)R², —C(O)H, —C(═S)R², —NHC(O)R², —NR²C(O)R², —NIIC(O)NH₂, —NR²C(O)NH₂, —NR²C(O)NHR², —NHC(O)NHR², —NR²C(O)N(R²)₂, —NHC(O)N(R²)₂, —CO₂H, —CO₂R², —NHCO₂R², —NR²CO₂R², —R², —CN, —NO₂, —NH₂, —NBR², —N(R²)₂, —NR²S(O)NH₂, —NR²S(O)₂NHR², —NH₂C(═NR²)NH₂, —N═C(NH₂)NH₂, —C(═NR²)NH₂, —NH—OH, —NR²—OH, —NR²—OR², —N═C═O, —N═C—S, —Si(R²)₃, —NH—NHR², —NHC(O)NHNH₂, NO, —N═C≡NR², and —S—CN, wherein each R² is independently alkyl (e.g., C₁-C₈ alkyl), aryl (e.g., C₆-C₁₂ aryl), or arylalkyl (e.g., C₇-C₁₄ aryl alkyl). In one embodiment, R² is C₁-C₈ alkyl. In another embodiment, R² is unsubstituted aryl, such as phenyl or pyridyl, or a substituted aryl, such as a substituted phenyl or a substituted pyridyl.

In one embodiment, each of R^{b} and R^{c} is independently selected from the group consisting of C₁-C₈ alkyl, C₁-C₈ alkoxy, aryl, aryl(C₁-C₈ alkyl), halogen, —NH₂, —NH(C₁-C₈ alkyl), —N(C₁-C₈ alkyl)₂, —CN, —C(═O)(C₁-C₈ alkyl), —(C═O)NH₂, —(C═O)NH(C₁-C₈ alkyl), —C(═O)N(C₁-C₈ alkyl)₂, —OH, —COOH, —COO(C₁-C₈ alkyl), —OCH(C₁-C₈ alkyl), —O(C═O)O(C₁-C₈ alkyl)-NO₂, —SH, —S(C₁-C₈ alkyl), —NH(C═O)(C₁-C₈ alkyl), —NH(C═O)O(C₁-C₈ alkyl), —O(C═O)NH(C₁-C₈ alkyl), —SO₂(C₁-C₈ alkyl), —NHSO₂(C₁-C₈ alkyl), and —SO₂NH(C₁-C₈ alkyl). In another embodiment, each R^{a} is independently selected from the group consisting of C₁-C₈ alkyl, C₁-C₈ alkoxy, phenyl, phenyl (C₁-C₈ alkyl), halogen, —CN, —NH₂, —NH(C₁-C₈ alkyl), —N(C₁-C₈ alkyl)₂, —(C═O)CH₃, —(C═O)NH₂, —OH, —COOH, —COO(C₁-C₈ alkyl), —OCH(C₁-C₈ alkyl), —O(C═O)O(C₁-C₈ alkyl), —NO₂, —SH, —S(C₁-C₈ alkyl), and —NH(C═O)(C₁-C₈ alkyl). In yet another embodiment, each R^{a} is independently selected from the group consisting of C₁-C₈ alkyl, C₁-C₈ alkoxy, phenyl, phenyl (C₁-C₈ alkyl), phenoxy, aryloxy, halogen, —CN, —NH₂, —NH-aryl, —(C═O)CH₃, —(C═O)NH₂, —OH, —COOH, —COO(C₁-C₈ alkyl), —OCH(C₁-C₈ alkyl), —COO-aryl, —OC(O)-aryl, —O(C═O)O(C₁-C₈ alkyl)-NO₂, —SH, —S(C₁-C₈ alkyl), —NH(C═O)(C₁-C₈ alkyl), and the like.

When q is 0, Z is a member selected from the group consisting of O, S, and NR^{d} wherein R^{d} is H or C₁-C₈ alkyl. When q is 1, Z is N. In one embodiment, the subscript q is 0, and Z is selected from the group consisting of O, S, and NH. In one instance, the subscript n is 0, 1 or 2. in one occurrence, Z is O or S. In another embodiment, the subscript q is 1. In one instance, L is CH₂ or SO₂.

Particular compounds of formula II are set forth in Table 2 below:

TABLE 2

| | |
|---|---|
| 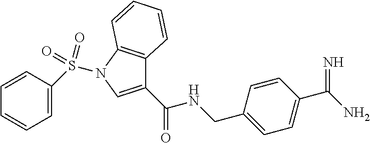 [SMILES: N=C(N)c1ccc(CNC(=O)c2cn(S(=O)(=O)c3ccccc3)c3ccccc23)cc1] | ASP-383 |
| 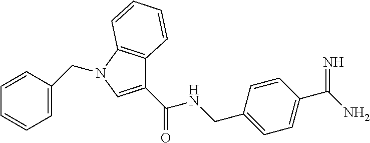 [SMILES: N=C(N)c1ccc(CNC(=O)c2cn(Cc3ccccc3)c3ccccc23)cc1] | ASP-381 |
| 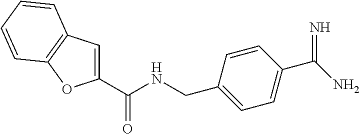 [SMILES: N=C(N)c1ccc(CNC(=O)c2cc3ccccc3o2)cc1] | ASP-485 |
| 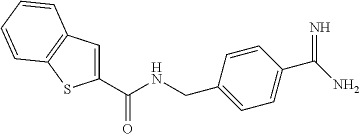 [SMILES: N=C(N)c1ccc(CNC(=O)c2cc3ccccc3s2)cc1] | ASP-486 |
| 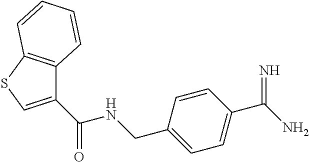 [SMILES: N=C(N)c1ccc(CNC(=O)c2csc3ccccc23)cc1] | ASP-491 |
| 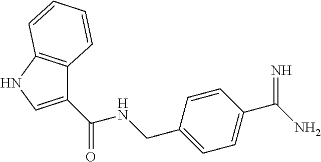 [SMILES: N=C(N)c1ccc(CNC(=O)c2c[nH]c3ccccc23)cc1] | ASP-493 |
| 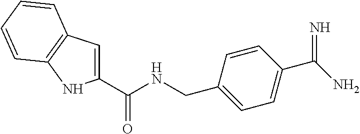 [SMILES: N=C(N)c1ccc(CNC(=O)c2cc3ccccc3[nH]2)cc1] | ASP-484 |

In one embodiment, the compound of formula I has a subformula IIa:

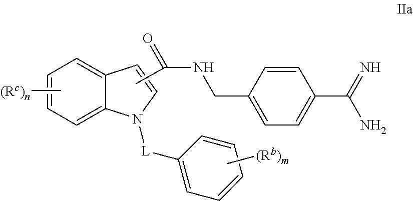

Substituents R^{b} and R^{c} and subscripts m are as defined above. In one instance, L is CH₂. In another instance, L is SO₂. In yet another instance, m is 0. In still another instance, n is 0.
In another embodiment, compounds of formula I have a subformula IIa-1:

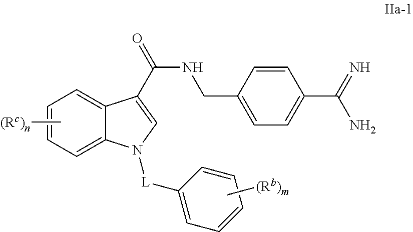

Table 3 provides compounds of PK inhibitors and their inhibition activities. The compound numbers correspond to numbers in Tables 1 and 2.

TABLE 3

| | | | Exper. | |
|---|---|---|---|---|
| | | Calc | Mass | PK Kᵢₐₚₚ |
| ASP- | Name | MW | (m + 1) | (μm) |
| 465 | 2,5-Dimethyl-1-pyridin-4- | 361.5 | 362.20 | 0.08 |
| | ylmethyl-1H-pyrrole-3-carboxylic | | | |
| | acid 4-carbamimidoyl-benzylamide | | | |
| 383 | 1-Benzenesulfonyl-1H-indole-3- | 432.5 | 433.10 | 0.17 |
| | carboxylic acid 4-carbamimidoyl- | | | |
| | benzylamide | | | |
| 381 | 1-Benzyl-1H-indole-3-carboxylic | 382.5 | 383.20 | 0.24 |
| | acid 4-carbamimidoyl-benzylamide | | | |
| 440 | 1-Benzyl-1H-pyrazole-4-carboxylic | 333.4 | 334.20 | 0.31 |
| | acid 4-carbamimidoyl-benzylamide | | | |
| 485 | Benzofuran-2-carboxylic acid 4- | 293.3 | | 0.87 |
| | carbamimidoyl-benzylamide | | | |
| 466 | 2-Methyl-5-phenyl-furan-3- | 333.4 | 334.10 | 1.29 |
| | carboxylic acid 4-carbamimidoyl- | | | |
| | benzylamide | | | |
| 445 | 1-Phenyl-1H-pyrazole-4-carboxylic | 319.4 | 320.10 | 1.43 |
| | acid 4-carbamimidoyl-benzylamide | | | |
| 486 | Benzo[b]thiophene-2-carboxylic | 309.4 | | 1.57 |
| | acid 4-carbamimidoyl-benzylamide | | | |
| 491 | Benzo[b]thiophene-3-carboxylic | 309.4 | | 2.11 |
| | acid 4-carbamimidoyl-benzylamide | | | |
| 493 | 1H-Indole-3-carboxylic acid 4- | 292.3 | | 2.45 |
| | carbamimidoyl-benzylamide | | | |
| 484 | 1H-Indole-2-carboxylic acid 4- | 292.3 | | 2.52 |
| | carbamimidoyl-benzylamide | | | |
| 558 | 1-(4-Bromo-benzyl)-5-methyl-1H- | 427.3 | 428.10 | 2.62 |
| | 1,2,3-triazole-4-carboxylic acid 4- | | | |
| | carbamimidoyl-benzylamide | | | |
| 523 | 5-Phenyl-thiophene-2-carboxylic | 335.4 | 336.10 | 7.66 |
| | acid 4-carbamimidoyl-benzylamide | | | |
| 559 | 1-(4-Fluoro-benzyl)-1H-1,2,3- | 352.4 | | 8.09 |
| | triazole-4-carboxylic acid 4- | | | |
| | carbamimidoyl-benzylamide | | | |
| 373 | 4-Methyl-2-phenyl-thiazole-5- | 350.4 | 351.10 | 8.87 |
| | carboxylic acid 4-carbamimidoyl- | | | |
| | benzylamide | | | |
| 525 | 5-Pyridin-2-yl-thiophene-2- | 336.4 | | 20.57 |
| | carboxylic acid 4-carbamimidoyl- | | | |
| | benzylamide | | | |
| 576 | 2,5-Dimethyl-1-phenyl-1H-pyrrole- | 346.4 | 347.1 | 0.04 |
| | 3-carboxylic acid 4- | | | |
| | carbamimidoyl-benzylamide | | | |
| 577 | 1-Benzyl-3,5-dimethyl-1H- | 361.5 | 362.1 | 1.13 |
| | pyrazole-4-carboxylic acid 4- | | | |
| | carbamimidoyl-benzylamide | | | |
| 578 | 1-(2-Chloro-benzyl)-3,5-dimethyl- | 395.9 | 396.1 | 1.64 |
| | 1H-pyrazole-4-carboxylic acid 4- | | | |
| | carbamimidoyl-benzylamide | | | |
| 579 | 5-Chloro-1-(2,6-dichloro-benzyl)- | 450.8 | 450.0 | 1.19 |
| | 3-methyl-1H-pyrazole-4-carboxylic | | | |
| | acid 4-carbamimidoyl-benzylamide | | | |
| 580 | 1-Benzyl-2,5-dimethyl-1H-pyrrole- | 360.5 | 361.1 | 0.03 |
| | 3-carboxylic acid 4- | | | |
| | carbamimidoyl-benzylamide | | | |
| 581 | 2,5-Dimethyl-1-(2,2,2-trifluoro- | 352.4 | 353.1 | 0.41 |
| | ethyl)-1H-pyrrole-3-carboxylic acid | | | |
| | 4-carbamimidoyl-benzylamide | | | |
| 582 | 1-(2-Chloro-benzyl)-1H-pyrazole- | 367.8 | 368.0 | 0.47 |
| | 4-carboxylic acid 4- | | | |
| | carbamimidoyl-benzylamide | | | |
| 583 | 5-Chloro-1-(4-fluoro-benzyl)-3- | 399.9 | 400.0 | 2.38 |
| | methyl-1H-pyrazole-4-carboxylic | | | |
| | acid 4-carbamimidoyl-benzylamide | | | |

In other embodiments, the plasma kallikrein inhibitor is a monoclonal antibody (e.g., MAB 13G11). In another embodiment, the subject is administered a second agent for reducing blood pressure within six months (e.g., within three months, 1 month, 2 weeks, 1 week, 3 days, 1 day, 12 hours, six hours, three hours, or one hour) of the plasma kallikrein inhibitor. The second agent may be selected from the group consisting of thiazide diuretics, beta blockers, angiotensin-converting enzyme (ACE) inhibitors, angiotensin II receptor blockers, calcium channel blockers, renin inhibitors, alpha blockers, alpha-beta blockers, and vasodilators (e.g., any described herein).

In another aspect, the invention features a kit including a plasma kallikrein inhibitor (e.g., any described herein) and instructions for administering the inhibitor to a patient for reducing blood pressure (e.g., to a patient suffering from hypertension or prehypertension).

In another aspect, the invention features a method for reducing blood pressure by administering an inhibitor of the kallikrein pathway, e.g., a kinin inhibitor, to a subject in need thereof.

The compounds described herein can be administered as frequently as necessary, including hourly, daily, weekly, or monthly. Frequency of dosage may also vary depending on the compound used and the particular disease treated. However, for treatment of most disorders, a dosage regimen of 4 times daily, 3 times daily, or less is preferred, with a dosage regimen of once daily or 2 times daily being particularly preferred. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient, in the context of the present invention, should be sufficient to effect a beneficial therapeutic response in the patient over time. The size of the dose also will be determined by the existence, nature, and extent of any adverse side effects that accompany the administration of a particular compound in a particular patient. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot, or via a patch.

The pharmaceutical compositions can be administered to the patient in a variety of ways, including topically, orally, parenterally, intravenously, intradermally, intramuscularly, colonically, rectally or intraperitoneally. Preferably, the pharmaceutical compositions are administered parenterally, topically, intravenously, intramuscularly or orally. The compounds of described herein can also be administered in combination with additional therapeutic agents or diagnostic agents.

In the above aspect of the invention, the patient may have increased BP as in essential hypertension. In another embodiment, the patient may have increased BP and additional risk factors of developing CVD, such as diabetes, or hyperlipidemia. In yet another embodiment, the subject may have increased BP and renal insufficiency, chronic kidney disease, or heart failure.

By “inhibitor of plasma kallikrein” is meant a compound that reduces the activity of plasma kallikrein and has an inhibitory constant, Kᵢ, no higher than 30 μM (e.g., less than 10, 1, or 0.1 μM). Methods for determining inhibitor constants are described, for example, in Example 1.

By “selective inhibitor of plasma kallikrein” is meant a compound that acts as an inhibitor of plasma kallikrein, at least 10-fold (e.g., at least 50, 100, 500), but does not significantly inhibit other forms of kallikrein (e.g., tissue kallikrein).

By “reducing blood pressure” is meant a reduction of at least 1 mm Hg (e.g., at least 2, 3, 4, 5, 6, 7, 8, 9, 10, 12, 15, 18, 20 mm Hg) of systolic blood pressure, diastolic blood pressure, or a combination thereof.

By “subject” is meant a human or non-human animal (e.g., a mammal).

In the generic descriptions of compounds of this invention, the number of atoms of a particular type in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups.

By “Cⱼ₋ₛ alkyl” is meant a branched or unbranched hydrocarbon group having from 1 to 8 carbon atoms. A C₁₋₉ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₈ alkyls include, without limitation, methyl, ethyl, n-propyl, isopropyl, cyclopropyl, cyclopropylmethyl, n-butyl, iso-butyl, sec-butyl, tert-butyl, cyclobutyl, pentyl, and cyclopentyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ arylalkyl” is meant an alkyl substituted by an aryl group (e.g., benzyl, phenethyl, or 3,4-dichlorophenethyl) having from 7 to 14 carbon atoms.

By “alkoxy” is meant a chemical substituent of the formula —OR, wherein R is selected from C₁₋₇ alkyl, C₂₋₇ alkenyl, C₂₋₇ alkynyl, C₂—₆ heterocyclyl, C₆₋₁₂ aryl, C₇₋₁₄ alkaryl, C₃₋₁₀ alkheterocyclyl, or C₁₋₇ heteroalkyl.

By “halide” or “halogen” is meant bromine, chlorine, iodine, or fluorine.

Other features and advantages of the invention will be apparent from the following Detailed Description, the drawings, and the claims.

### BRIEF DESCRIPTION OF THE DRAWINGS

FIG. 1 is a graph showing the time course of systolic blood pressure (SBP) measured using telemetry in rats infused with Ang-II in the absence or presence of ASP-440. * and † indicate p<0.05 for Day 0 vs Ang-II alone (n=4) and Ang-II+ASP-440 (n=7), respectively. ‡ indicates p<0.05 for Ang-II alone vs Ang-Il⁺ ASP-440. NS indicates statistically non-significant change from Day 0.

FIGS. 2A and 2B are graphs showing normalized retinal blood flow (FIG. 2A) and increased blood vessel diameter (FIG. 2B) following ASP-440 administration in diabetic rats.

### DETAILED DESCRIPTION

We have shown that the exemplary plasma kallikrein inhibitor, ASP-440, is capable of reducing blood pressure in rats that have been treated with Angiotensin-II. Based on these data, the present invention features methods of reducing blood pressure in a subject in need thereof by administering a plasma kallikrein inhibitor. As explained herein, any plasma kallikrein inhibitor known in the art can be used in the present invention.

### Plasma Kallikrein Inhibitors

Subjects in need of blood pressure reduction can be administered any inhibitor of plasma kallikrein known in the art. Certain inhibitors are described in Formula I and Formula II herein and in Tables 1, 2, and 3 and are also described in PCT Publication No. WO 2008/016883. Other exemplary inhibitors include DX-88, Bz-Pro-Phe-boroArg (Stadnicki et al., Dig Dis Sci 41:912-20, 1996), CU-2010 (Dietrich et al., Anesthesiology 110:123-30, 2009), PKSI-527 (Enzo Life Sciences, Farmingdale, N.Y.), Arg15-aprotinin (Scott et al., Blood 69:1431-6, 1987), Cl-inhibitor, a2-macroglobulin, 2′S,2″R)-4-(2′-(2″(carboxymethylamino)-3″-cyclohexyl-propanoylamino)-3′-phenyl-propanoylamino)piperidine-1-carboxamidin (FE999026, CH-4215; Griesbacher et al., Br J Pharmacol 137:692-700, 2002), AdKI (González et al., Toxicon 43:219-23, 2004), trans-4-aminomethylcyclohexanecarbonylphenylalanine-4-carboxyan ilide (Tsuda et al., Chem Pharm Bull (Tokyo) 46:452-7, 1998), or a Kunitz-type serine proteinase inhibitor. Exemplary Kunitz-type serine proteinase inhibitors include BmTlsint and BmTlsint Mut (Sasaki et al., Biochem Biophys Res Commun 341:266-72, 2006), thefirst Kunitz domain (KD1) from the physiological inhibitor hepatocyte growth factor activator inhibitor 1B (HAI-1B) (Shia et al., J Mol Biol 346:1335-49, 2005), and those described in U.S. Pat. Nos. 5,780,265, 5,786,328, 5,795,865, 6,057,287, and 6,333,402.

Still other plasma kallikrein inhibitors include bicyclic peptides, such as those described by Heinis et al., Nat Chem Biol 5:502-507, 2009. Such peptides include any of PK 1-P1(23 described in this reference.

### Subjects in Need of Blood Pressure Reduction

Inhibitors of plasma kallikrein can be administered to any subject that needs or desires a reduction in blood pressure. In certain embodiments, the subject is suffering from hypertension, which is typically defined as having a systolic blood pressure (SBP) of 140 mm Hg or greater and a diastolic blood pressure (DBP) of 90 mm Hg or greater. It has been shown that even mild increases in blood pressure over normal (120/80) increase the risk of cardiovascular disease. Subjects with SBP between 120 and 139 mm Hg or DBP between 80 and 89 mm Hg are considered to have pre-hypertension; such subjects may also be treated in the methods of the present invention. Subjects suffer from primary or essential hypertension when no cause of hypertension can be identified. In cases where the subject's hypertension is caused by a condition, habit, or medication, this is referred to as secondary hypertension. Secondary hypertension can result from, for example, an adrenal gland tumor, alcohol abuse, anxiety and stress, arteriosclerosis, birth control pills, coarctation of the aorta, cocaine use, cushing syndrome, diabetes, kidney disease (e.g., glomerulonephritis (inflammation of kidneys)); kidney failure, renal artery stenosis, and renal vascular obstruction or narrowing), medications (e.g., appetite suppressants, certain cold medications, corticosteroids, and migraine medications), hemolytic-uremic syndrome, henoch-Schonlein purpura, obesity, pain, periarteritis nodosa, pheochromocytoma, pregnancy (called gestational hypertension), primary hyperaldosteronism, renal artery stenosis, retroperitoneal fibrosis, and Wilms' tumor.

In other cases, the subject has, or is at increased risk of developing, angioedema. Subjects receiving ACE inhibitors, for example, are at increased risk for angioedema and thus may be administered a plasma kallikrien inhibitor, as described herein.

### Prodrugs and Other Modified Plasma Kallikrein Inhibitors

In some cases, a compound that is effective in vitro in inhibiting plasma kallikrein is not an effective therapeutic agent in vivo. For example, this could be due to low bioavailability of the compound. One way to circumvent this difficulty is to administer a modified drug, or prodrug, with improved bioavailability that converts naturally to the original compound following administration. Such prodrugs may undergo transformation before exhibiting their full pharmacological effects. Prodrugs contain one or more specialized protective groups that are specifically designed to alter or to eliminate undesirable properties in the parent molecule. In one embodiment, a prodrug masks one or more charged or hydrophobic groups of a parent molecule. Once administered, a prodrug is metabolized in vivo into an active compound.

Prodrugs may be useful for improving one or more of the following characteristics of a drug: solubility, absorption, distribution, metabolization, excretion, site specificity, stability, patient acceptability, reduced toxicity, or problems of formulation. For example, an active compound may have poor oral bioavailability, but by attaching an appropriately-chosen covalent linkage that may be metabolized in the body, oral bioavailability may improve sufficiently to enable the prodrug to be administered orally without adversely affecting the parent compound's activity within the body.

A prodrug may be carrier-linked, meaning that it contains a group such as an ester that can be removed enzymatically. Optimally, the additional chemical group has little or no pharmacologic activity, and the bond connecting this group to the parent compound is labile to allow for efficient in vivo activation. Such a carrier group may be linked directly to the parent compound (bipartate), or it may be bonded via a linker region (tripartate). Common examples of chemical groups attached to parent compounds to form prodrugs include esters, methyl esters, sulfates, sulfonates, phosphates, alcohols, amides, imines, phenyl carbamates, and carbonyls.

As one example, methylprednisolone is a poorly water-soluble corticosteroid drug. In order to be useful for aqueous injection or ophthalmic administration, this drug must be converted into a prodrug of enhanced solubility. Methylprednisolone sodium succinate ester is much more soluble than the parent compound, and it is rapidly and extensively hydrolysed in vivo by cholinesterases to free methylprednisolone.

Caged compounds may also be used as prodrugs. A caged compound may have, e.g., one or more photolyzable chemical groups attached that renders the compound biologically inactive. In this example, flash photolysis releases the caging group (and activates the compound) in a spatially or temporally controlled manner. Caged compounds may be made or designed by any method known to those of skill in the art.

For further description of the design and use of prodrugs, see Testa and Mayer, Hydrolysis in Drug and Prodrug Metabolism: Chemistry, Biochemistry and Enzymology, published by Vch. Verlagsgesellschaft Mbh. (2003).

Other modified compounds are also possible in the methods of the invention. For example, a modified compound need not be metabolized to form a parent molecule. Rather, in some embodiments, a compound may contain a non-removable moiety that, e.g., increases bioavailability without substantially diminishing the activity of the parent molecule. Such a moiety could, for example, be covalently-linked to the parent molecule and could be capable of translocating across a biological membrane such as a cell membrane, in order to enhance cellular uptake. Exemplary moieties include peptides, e.g., penetratin or TAT. An exemplary penetratin-containing compound according to the invention is, e.g., a peptide comprising the sixteen amino acid sequence from the homeodomain of the Antennapedia protein (Derossi et al., J. Biol. Chem. 269:10444-10450, 1994), particularly a peptide having the amino acid sequence RQIKIWFQNRRMKWKK, or including a peptide sequence disclosed by Lin et al. (J. Biol. Chem. 270:14255-14258, 1995). Others are described in U.S. Patent Application Publication No. 2004-0209797 and U.S. Pat. Nos. 5,804,604, 5,747,641, 5,674,980, 5,670,617, and 5,652,122. In addition, a compound of the invention could be attached, for example, to a solid support.

### Additional Treatments for Reducing Blood Pressure

In addition to the plasma kallikrein inhibitors described herein, the subject may be administered any other medication known in the art that reduces blood pressure. Such therapies include thiazide diuretics (e.g., bendroflumethiazide, chlorothiazide, chlorthalidone, hydrochlorothiazide, hydroflumethiazide, methyclothiazide, metolazone, polythiazide, quinethazone, and trichlormethiazide, indapamide (Lozol)), beta blockers (e.g., propanolol, nadolol, timolol, pindolol, labetolol, metoprolol, atenolol, esmolol, acebutolol, carvedilol, bopindolol, carteolol, oxprenolol, penbutolol, medroxalol, bucindolol, levobutolol, metipranolol, bisoprolol, nebivolol, betaxolol, celiprolol, solralol, and propafenone), angiotensin-converting enzyme (ACE) inhibitors (e.g., benazepril, captopril, cilazapril, enalapril, fosinopril, lisinopril, moexipril, perindopril, quinapril, ramipril, spirapril, temocapril, and trandolapril), angiotensin II receptor blockers (e.g., candesartan, eprosartan, irbesartan, losartan, and valsartan), calcium channel blockers (e.g., amlodipine, amlodipine/benzapril, bepridil, diltiazem, felodipine, imidapril, isradipine, isosorbide, manidipine, nicardipine, nifedipine, nilvadipine, nimodipine, nisoldipine, and verapamil), renin inhibitors (e.g., Aliskiren (Tekturna) and remikiren), alpha blockers (e.g., chlorthalidone, clonidine, doxazosin, guanzbenz, guanadrel, guanethidine, guanfacine, methyldopa, phenoxybenzamine, polythiazide/prazosin, prazosin, reserpine, and terazosin), alpha-beta blockers (e.g., bucindolol, carvedilol and labetalol), central-acting agents (e.g., clonidine, guanfacine, and methyldopa), and vasodilators (e.g., clonidine, doxazosin, guanabenz, guanfacine, hydralazine, methyldopa, minoxidil, prazosin, terazosin, darusentan, pinacidil, sodium nitroprusside, fenodolpam, diazoxide, alprostadil, amyl nitrate, cilostazol, cyclandelate, ethaverine, flosequinan, isoxsuprine, nitroglycerin, papaverine, pentoxifyline, and tolazoline).

### Formulation of Pharmaceutical Compositions

The plasma kallikrein inhibitor used in the invention (e.g., a compound of Formula I or II, such as ASP-440 or ASP-465) may be formulated and administered by any suitable means that results in a concentration of the compound that is able to reduce blood pressure. The compound may be contained in any appropriate amount in any suitable carrier substance, and is generally present in an amount of 1-95% by weight of the total weight of the composition. The composition may be provided in a dosage form that is suitable for oral, parenteral (e.g., intravenously or intramuscularly), rectal, cutaneous, nasal, vaginal, inhalant, skin (patch), ocular, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agents of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

### Parenteral Compositions

A composition containing a plasma kallikrein inhibitor (e.g., a compound of Formula I or II, such as ASP-440 or ASP-465) may be administered parenterally by injection, infusion, or implantation (intraocular, subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

In some embodiments, the composition is especially adapted for administration into or around the eye. For example, a composition can be adapted to be used as eye drops, or injected into the eye, e.g., using peribulbar or intravitreal injection. Such compositions should be sterile and substantially endotoxin-free, and within an acceptable range of pH. Certain preservatives are thought not to be good for the eye, so that in some embodiments a non-preserved formulation is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev Ind Pharm. 2002 May; 28(5):473-93; Clinical Ocular Pharmacology, Bartlett et al. (Butterworth-Heinemann; 4th edition (Mar. 15, 2001)); and Ophthalmic Drug Delivery Systems (Drugs and the Pharmaceutical Sciences: a Series of Textbooks and Monographs), Mitra (Marcel Dekker; 2nd Rev&Ex edition (Mar. 1, 2003)).

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in a form suitable for sterile injection. To prepare such a composition, the suitable active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Dosages

The dosage of any plasma kallikrein inhibitor (e.g., a compound of formula I and formula II, such as ASP-440 or ASP-465) depends on several factors, including: the administration method, the condition to be treated or the biological effect desired, the severity of the condition, whether the condition is to be treated or prevented, and the age, weight, and health of the subject to be treated.

With respect to the treatment methods of the invention, it is not intended that the administration of a compound to a subject be limited to a particular mode of administration, dosage, or frequency of dosing; the present invention contemplates all modes of administration, including intramuscular, intraocular, intravenous, intraperitoneal, intravesicular, intraarticular, intralesional, subcutaneous, or any other route sufficient to provide a dose adequate to achieve the desired biological or therapeutic effect. The compound may be administered to the subject in a single dose or in multiple doses. For example, a compound described herein or identified using screening methods of the invention may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compound. For example, the dosage of a compound can be increased if the lower dose does not provide sufficient biological activity (e.g., in the treatment of a disease or condition described herein). Conversely, the dosage of the compound can be decreased, for example, if the disease or condition is reduced or eliminated.

While the attending physician ultimately will decide the appropriate amount and dosage regimen, a therapeutically effective amount of a compound described herein (e.g., ASP-440 or ASP-465), may be, for example, in the range of 0.0035 μg to 20 μg/kg body weight/day or 0.010 μg to 140 μg/kg body weight/week. Desirably a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In addition, a therapeutically effective amount may be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. Furthermore, a therapeutically effective amount of a compound may be, for example, in the range of 100 μg/m² to 100,000 μg/m² administered every other day, once weekly, or every other week. In a desirable embodiment, the therapeutically effective amount is in the range of 1000 μg/m² to 20,000 μg/m², for example, at least 1000, 1500, 4000, or 14,000 μg/m² of the compound administered daily, every other day, twice weekly, weekly, or every other week.

The following examples are intended to illustrate, rather than limit, the invention.

### Example 1

### Determination of Inhibitory Activity of a Compound towards Plasma Kallikrein

Human plasma kallikrein (PK) was obtained from Haemtech Technologies (Essex Junction, Vt.). The enzymatic activity of PK was assayed using the synthetic peptide substrate H-D-Pro-Phe-Arg-pNA (Bachem, Inc., Switzerland) with the cleavage of the substrate by the enzyme resulting in an increase in A₄₀₅, measured using a Molecular Devices Vₘₐₓ Kinetic Microplate Reader. The uninhibited (control) activity of PK was determined by adding 190 μl of PK solution (1 nM in 0.05 M HEPES, pH 7.5, 0.01% Triton X-100) to 10 μl of H-D-Pro-Phe-Arg-pNA (2 mM in DMSO) in individual microtiter plate wells, mixed immediately by shaking, and the rate of increase in A₄₀₅ (rate of substrate cleavage) determined over 120-180 sec. In parallel, compounds of the present invention were mixed in separate wells with the synthetic substrate to attain final concentrations of between 0.01-30 μM in the final 200 μl reaction mixture, and the reaction initiated by the addition of 190 μl of the PK solution. A diminished rate of increase in A₄₀₅ in the presence of a compound denotes inhibition of PK activity, and the apparent inhibition constant of the interaction can be determined by using the following equation

K_{i,app}=[I]/(PK_{Control}/PK₁−1)

where [I]=concentration of inhibitory compound, PK_{Control}=rate of substrate cleavage by uninhibited PK, PK₁=rate of substrate cleavage by PK in the presence of inhibitory compound.

The corrected Kᵢ of the interaction can be obtained as follows from the calculated K_{i,app}

Kᵢ=K_{i,app}/([S]/Kₘ+1)

where [S]=concentration of synthetic substrate, and Kₘ Michaelis constant of synthetic substrate for PK, in this case determined experimentally to be 0.15 mM under these conditions.

Examples of Kᵢₐₚₚ values for PK inhibitors are provided in Table 3.

### Example 2

### Demonstration of BP-Lowering Effect of PK Inhibitor in Ang-II-Induced Rat Hypertension Model

Rats were infused continuously with Ang-II, and concurrently with either ASP-440 or vehicle control. Treatments were achieved by the use of subcutaneous implantation of two sets of Alzet mini-osmotic pumps (DURECT corporation, Cupertino Calif.), one containing Ang-II, the other containing either ASP-440 or vehicle control. Angll (EMD Chemicals Inc, La Jolla, Calif.) was delivered at 300 ng/kg/min. ASP-440 was delivered at 16 ƒg/kg/hr, and control pumps were filled with vehicle (10% polyethylene glycol, 90% phosphate-buffered saline).

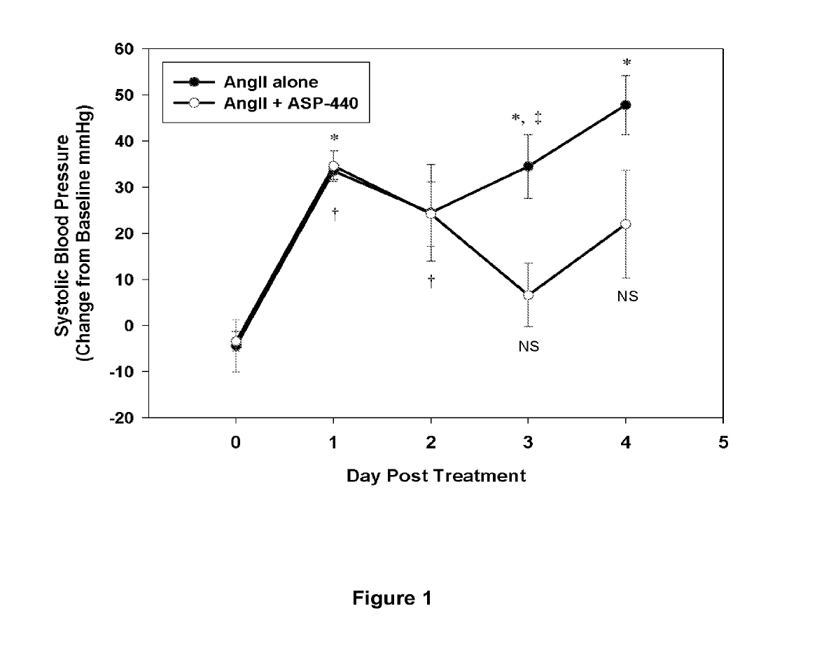

Blood pressure measurements by telemetry were performed using PA-C40 transmitters (Data Sciences International, St. Paul, Minn.). Under anesthesia, a telemetric transmitter was fixed to the interscapular area and the pressure sensing catheter was inserted via the external carotid into the common carotid with the tip approximately 3 mm distal to the aortic junction. Rats were housed individually on a receiver pad and blood pressure was monitored continuously. Systolic and diastolic pressure was averaged over a 15 second intervals every 15 minutes over a fixed 4 h time-interval on each day. Baseline readings (Day 0) were obtained 48 hours after catheter implantation, following implantation of Alzet mini-pumps. Results from these experiments are shown in FIG. 1.

### Example 3

### ASP-440 Normalizes Blood Flow and Increases Blood Vessel Diameter

We examined the effect of ASP-440 on retinal vessel diameters, mean circulation times (MCT), and retinal blood flow (RBF) in rats with diabetes. Diabetes was induced in Sprague Dawley rats with an intraperitoneal injection of 55 mg/kg of streptozotocin (STZ) (Sigma, St. Louis, Mo., USA) in 10 mmol/l citrate buffer, pH 4.5 after a 12 h fast. Diabetes was confirmed with blood glucose measurements (>14 mmol/l) 24 h after STZ injection. The rats were housed under standard conditions with free access to water and standard food. Two weeks after diabetes onset rats were implanted with a subcutaneous osmotic model 2002 Alzet pump containing either 12 mg/ml ASP-440 or saline vehicle. Rats were infused for 2 weeks at a rate of 0.5 μg/hr. Retinal vessel diameters and MCT was measured after 4 weeks of total diabetes duration and RBF was calculated as described by Horio et al. (Diabetologia 47:113-23, 2004).

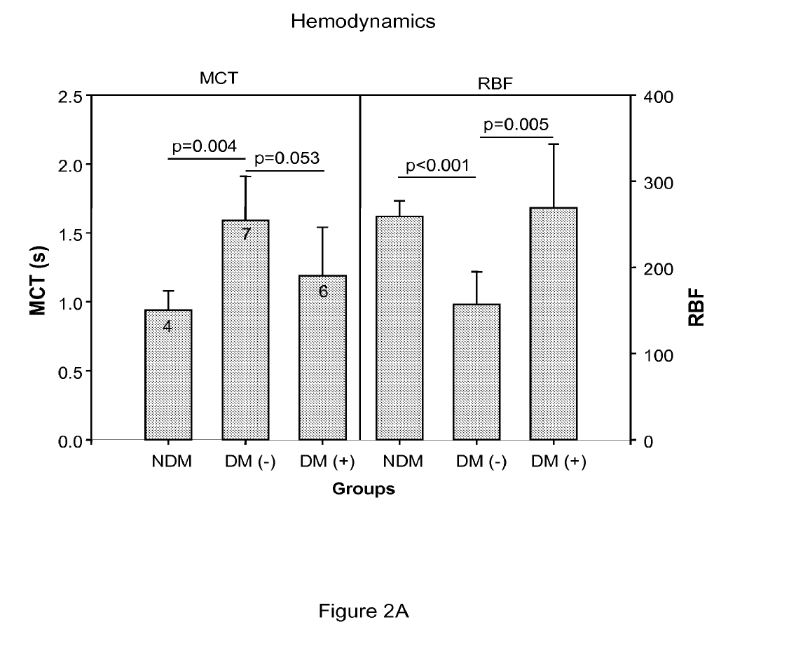

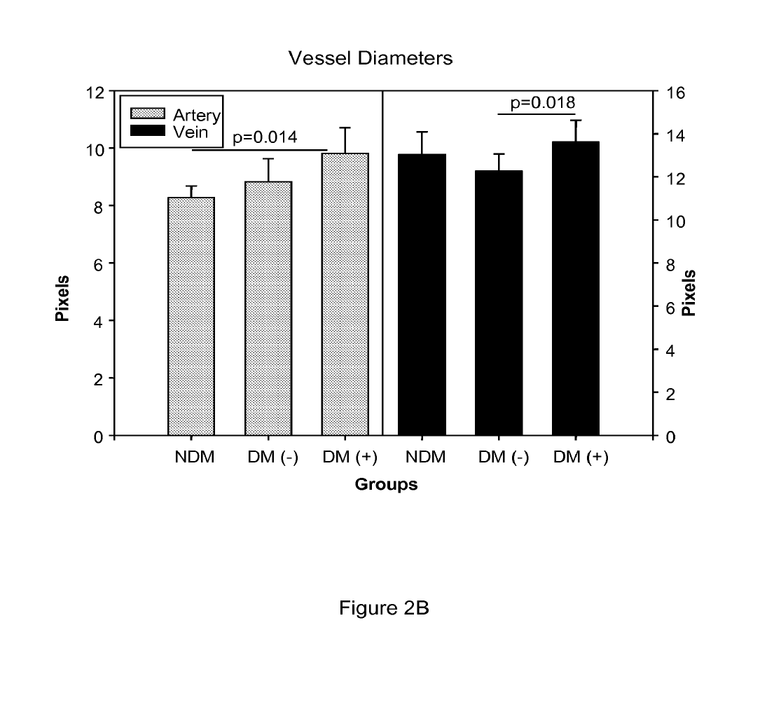

We show that MCT is prolonged and RBF is reduced in diabetic rats compared with age matched nondiabetic controls (FIG. 2A). These changes in retinal hemodymanics in diabetes are consistent with previous reports (Florio et al, supra). Diabetic rats receiving the plasma kallikrein inhibitor ASP-440 displayed shortened MCT and increased RBF compared with diabetic rats receiving vehicle control. ASP-440 treatment was also associated with increased retinal artery and vein diameters compared with nondiabetic rats and saline-treated diabetic rats (FIG. 2B). These findings demonstrate that the plasma kallikrein inhibitor exerts multiple effects on the retinal vasculature, including vasodilation of primary vessels and reduced microvascular resistance, measured as an increase in MCT.

To make the measurements described above, the following procedure was used. Immediately before video fluorescein angiography (VFA) measurements, each rat was anaesthetized, the left eye was dilated (1% tropicamide, Mydriacyl: Alcon, Fort Worth, Tex., USA), and a 100 μl syringe (Hamilton, Reno, Nev., USA) containing 10% sodium fluorescein was connected to the externalized jugular vein catheter. The rats were positioned on a platform attached to a scanning laser ophthalmoscope (SLO, Rodenstock Instrument, Munich, Germany) to image the fundus. The optic disc was centered and focused in the field of view, the VFA recording sequence was initiated, and a 5 μl bolus of fluorescein dye was rapidly injected into the jugular vein catheter. The injection time was marked on the video recording. The recorded fluorescein angiograms were digitized on a frame-by-frame basis and analyzed densitometrically to determine retinal vessel diameters and retinal mean circulation times (MCTs), as described by Horio et al. (supra). Sample sites were chosen using primary retinal vessels at a fixed (1 optic disc diameter) radial distance from the centre of the optic disc. Vessel diameters in units of pixels were determined during peak fluorescein arterial and venous filling times at the defined vessel sample sites using a boundary-crossing algorithm. The average diameter for each vessel was measured for each sample site. The average vessel diameters for each eye represent the average of the individual vessel diameters for that eye. At the fixed vessel sites, the average vessel fluorescence within a sample area defined by the vessel width was measured on a frame-by-frame basis to generate temporal fluorescence intensity or dye dilution curves. The resultant artery and vein fluorescence data were fit to a log normal distribution function from which average arterial and venous circulation times were calculated. The arterial appearance time (AT) of the dye bolus, defined as the time between dye injection and the first detectable appearance (vessel fluorescence intensity greater than background level by 2 times the standard deviation of the average background intensity) of dye in the retinal artery, represents an assessment of systemic circulation times. The average MCT was calculated as the difference between the average retinal mean arterial and venous filling times for all primary arteries and veins. Retinal blood flow was calculated by dividing the sum of the squares of the arterial and venous diameters by the MCT.

### Other Embodiments

All patents, patent applications (including U.S. Provisional Application No. 61/200,600, filed Dec. 2, 2008), and publications mentioned in this specification are herein incorporated by reference to the same extent as if each independent patent, patent application, or publication was specifically and individually indicated to be incorporated by reference.

What is claimed is:

1. A method for reducing blood pressure in a subject in need thereof, said method comprising administering to said subject an effective amount of an inhibitor of plasma kallikrein.

2. The method of claim 1, wherein the subject is a human.

3. The method of claim 2, wherein the systolic blood pressure (SBP) of said subject is greater than 125 mm Hg.

4. The method of claim 3, wherein said SBP is greater than 139 mm Hg.

5. The method of claim 4, wherein the diastolic blood pressure of said subject is greater than 89 mm Hg.

6. The method of claim 1, wherein said subject is suffering from primary hypertension.

7. The method of claim 1, wherein said subject is suffering from secondary hypertension.

8. The method of claim 1, wherein said subject is prehypertensive.

9. The method of claim 1, wherein said subject has, or is at increased risk of having, angioedema.

10. The method of claim 9, wherein said subject has been previously treated with at least on angiotensin-converting enzyme (ACE inhibitor).

11. The method of claim 1, wherein the plasma kallikrein inhibitor is a selective plasma kallikrein inhibitor.

12. The method of claim 1, wherein the inhibitor is a naturally occurring compound.

13. The method of claim 12, wherein the inhibitor is the naturally occurring protein Cl-Inhibitor.

14. The method of claim 1, wherein the inhibitor is a non-naturally occurring compound.

15. The method of claim 14, wherein the inhibitor is ecallantide (DX-88).

16. The method of claim 14, wherein the inhibitor is a bicyclic peptide.

17. The method of claim 16, wherein said bicyclic peptide is selected from the group consisting of PK1-PK23.

18. The method of claim 14, wherein the inhibitor has the formula I: 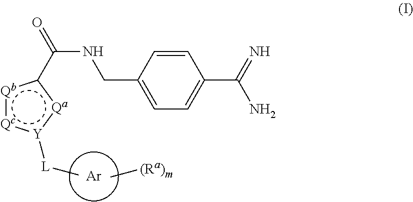
wherein Ar is a bond or an aromatic ring selected from the group consisting of benzene, pyridine, and pyrimidine;
the subscript m is an integer of from 0 to 5;
each R^{a} is independently selected from the group consisting of cycloalkyl, (C₁-C₈)haloalkyl, halogen, —OH, —OR¹, —OSi(R¹)₃, —OC(O)O—R¹, —OC(O)R¹, —OC(O)NHR¹, —OC(O)N(R¹)₂, —SH, —SR¹, —S(O)R¹, —S(O)₂R¹, —SO₂NH₂, —S(O)₂NHR¹, —S(O)₂N(R¹)₂, —NHS(O)₂R¹, —NR¹S(O)₂R¹, —C(O)NH₂, —C(O)NHR¹, —C(O)N(R¹)₂, —C(O)R¹, —C(O)H, —NHC(O)R′, —NR¹C(O)R¹, —NHC(O)NH₂, —NR′C(O)NH₂, —NR′C(O)NHR¹, —NHC(O)NHR¹, —NR¹C(O)N(R¹)₂, —NHC(O)N(R¹)₂, —CO₂H, —CO₂R¹, —NHCO₂R¹, —NR′CO₂R¹, —R¹, —CN, —NO₂, —NH₂, —NHR¹, —N(R¹)₂, —NR¹S(O)NH₂, —NR¹S(O)₂NHR¹, —NH₂C(═NR¹)NH₂, —N═C(NH₂)NH₂, —C(═NR¹)NH₂, —NH—OH, —NR¹—OH, —NR¹—OR¹, —N═C═O, —N═C═S, —Si(R¹)₃, —NH—NHR¹, —NHC(O)NHNH₂, NO, —N═C≡NR¹, and —S—CN, wherein each R′ is independently alkyl, aryl, or arylalkyl;
L is a linking group selected from the group consisting of a bond, CH₂ and SO₂;
Q^{a}, Q^{b}, and Q^{c} are each members independently selected from the group consisting of N, S, O, and C(R^{q}) wherein each R^{q} is independently selected from the group consisting of H, C₁₋ₛ alkyl, halo, and phenyl, and the ring having Q^{a}, Q^{b}, Q^{c}, and Y as ring vertices is a five-membered ring having two double bonds;
Y is a member selected from the group consisting of C and N;
when Ar is a bond, m is 1;
when Ar is an aromatic ring, m is an integer of from 0-5; or a pharmaceutically acceptable salt thereof.

19. The method of claim 18, wherein Ar is an aromatic ring selected from the group consisting of benzene, pyridine, and pyrimidine.

20. The method of claim 18, wherein Ar is a bond and m is 1.

21. The method of claim 18, wherein said compound has the formula Ia:

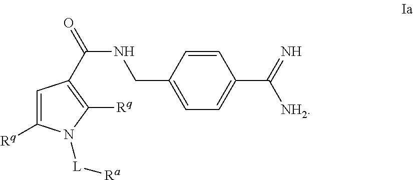

22. The method of claim 21, wherein L is a bond and Y is N.

23. The method of claim 18, wherein L is a bond, Y is N and Ar is a benzene ring.

24. The method of claim 18, wherein Q^{a}, Q^{b}, and Q^{c} are each independently C(R^{q}).

25. The method of claim 18, wherein Q^{b} is N.

26. The method of claim 18, wherein Y is C; Q^{a} is S, and Ar is selected from the group consisting of phenyl and pyridyl.

27. The method of claim 26, wherein Q^{c} is C.

28. The method of claim 18, wherein L is CH₂ and Y is N.

29. The method of claim 28, wherein Q^{a} is C.

30. The method of claim 29, wherein Q^{b} and Q^{c} are each independently selected from the group consisting of N and C(R^{q}).

31. The method of claim 30, wherein Ar is benzene or pyridine.

32. The method of claim 18, wherein L is a bond and Y is C.

33. The method of claim 32, wherein Q^{b} is O; and Q⁸ and 0⁸ are each C(R^{q}).

34. The method of claim 18, wherein L is SO₂ and Y is N.

35. The method of claim 18, wherein each R^{a} is independently selected from the group consisting of C₁-C₈ alkyl, C₁-C₈ alkoxy, aryl, aryl(C₁-C₈ alkyl), halogen, —NH₂, —NH(C₁-C₈ alkyl), —N(C₁-C₈ alkyl)₂, —CN, —C(═O)(C₁-C₈ alkyl), —(C═O)NH₂, —(C═O)NH(C₁-C₈ alkyl), —C(═O)N(C₁-C₈ alkyl)₂, —OH, —COOH, —COO(C₁-C₈ alkyl), —OCH(C₁-C₈ alkyl), —O(C═O)O(C₁-C₈alkyl)-NO₂, —SH, —S(C₁-C₈ alkyl), —NH(C═O)(C₁-C₈ alkyl), —NH(C═O)O(C₁-C₈—O(C═O)NH(C₁-C₈ alkyl), —SO₂(C₁-C₈ alkyl), —NHSO₂(C₁-C₈ alkyl), and —SO₂NH(C₁-C₈ alkyl).

36. The method of claim 35, wherein each R^{a} is independently selected from the group consisting of C₁-C₈ alkyl, C₁-C₈ alkoxy, phenyl, phenyl (C₁-C₈ alkyl), halogen, —CN, —NH₂, —NH(C₁-C₈ alkyl), —N(C₁-C₈ alkyl)₂, —(C═O)CH₃, (C═O)NH₂, —OH, —COOH, —COO(C₁-C₈ alkyl), —OCH(C₁-C₈ alkyl), —O(C═O)O(C₁-C₈ alkyl), —NO₂, —SH, —S(C₁-C₈ alkyl), and —NH(C═O)(C₁-C₈ alkyl).

37. The method of claim 36, wherein R^{a} is halogen.

38. The method of claim 18, wherein said compound is selected from the group consisting of:

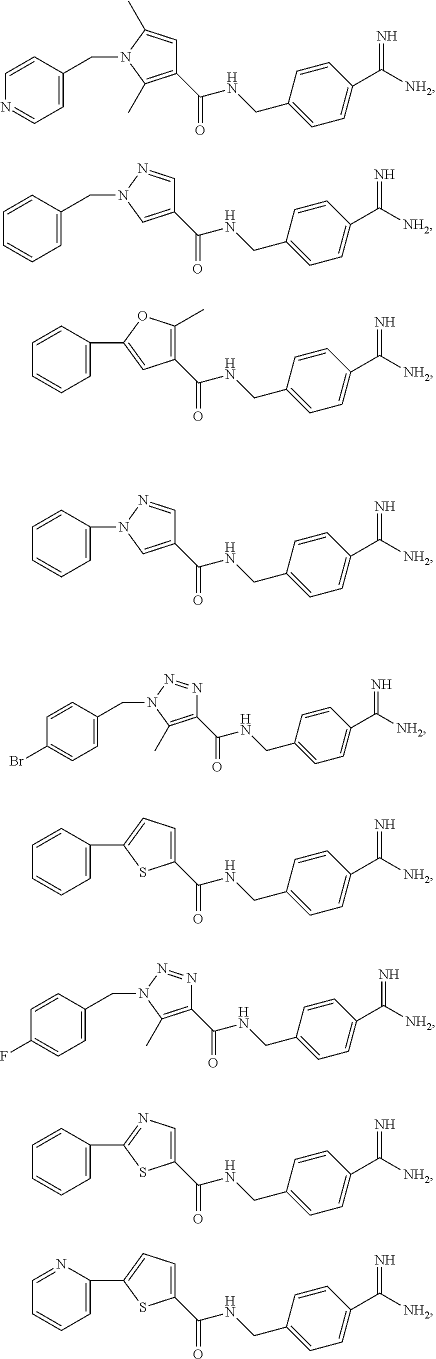

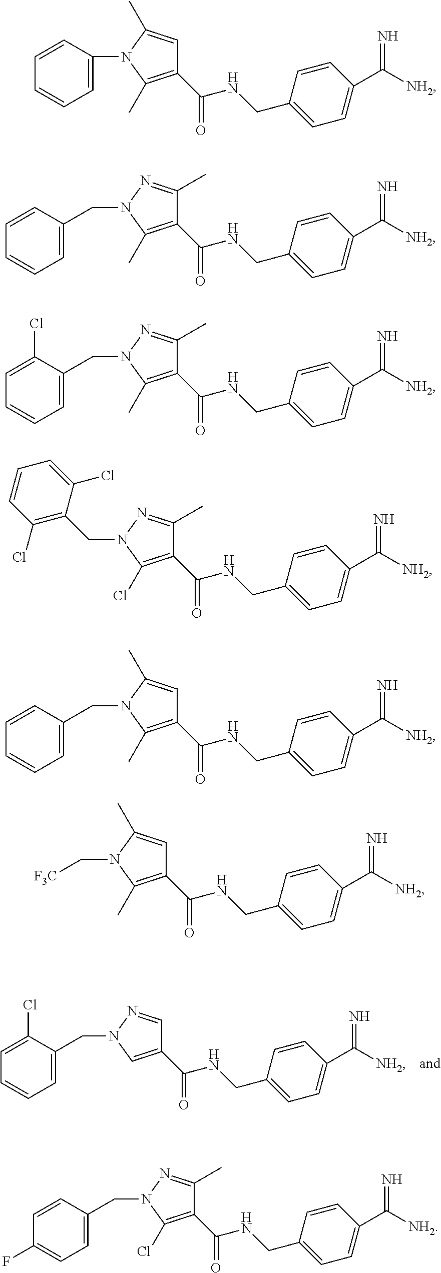

39. A method of reducing blood pressure in a subject in need thereof, said method comprising administering to said subject an effective amount of a compound having the formula II: 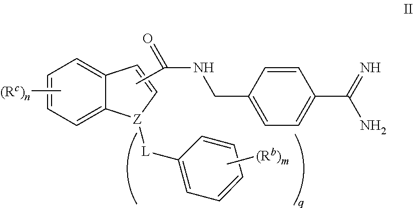
wherein
the subscript m is an integer of from 0 to 5;
the subscript n is an integer of from 0 to 4;
the subscript q is an integer of from 0 to 1;
L is a linking group selected from the group consisting of a bond, CH₂, and SO₂;
each of R^{h} and R^{c} is independently selected from the group consisting of cycloalkyl, (C₁-C₈)haloalkyl, halogen, —OH, —OR², —OSi(R²)₃, —OC(O)O—R², —OC(O)R², —OC(O)NHR², —OC(O)N(R²)₂, —SH, —SR², —S(O)R², —S(O)₂R², —SO₂NH₂, —S(O)₂NHR², —S(O)₂N(R²)₂, —NHS(O)₂R², —NR²S(O)₂R², —C(O)NH₂, —C(O)NHR², —C(O)N(R²)₂, —C(O)R², —C(O)H, —C(═S)R², —NHC(O)R², —NR²C(O)R², —NHC(O)NH₂, —NR²C(O)NH₂, —NR²C(O)NHR², —NHC(O)NHR², —NR²C(O)N(R²)₂, —NHC(O)N(R²)₂, —CO₂H, —CO₂R², —NHCO₂R², —NR²CO₂R², —R², —CN, —NO₂, —NH₂, —NHR², —N(R²)₂, —NR²S(O)NH₂, —NR²S(O)₂NHR², —NH₂C(═NR²)NH₂, —N═C(NH₂)NH₂, —C(═NR²)NH₂, —NH—OH, —NR²—OH, —NR²—OR², —N═C═O, —N═C═S, —Si(R²)₃, —NH—NHR², —NHC(O)NHNH₂, NO, —N═C≡NR², and —S—CN, wherein each R² is independently alkyl, aryl, or arylalkyl;
when q is 0, Z is a member selected from the group consisting of O, S, and NR^{d}, wherein R^{d} is H or C₁-C₈ alkyl;
when q is 1, Z is N; or
a pharmaceutically acceptable salt thereof.

40. The method of claim 39, wherein the subscript q is 0 and Z is selected from the group consisting of O, S, and NH.

41. The method of claim 40, wherein the subscript n is an integer of from 0 to 2.

42. The method of claim 41, wherein Z is O or S.

43. The method of claim 39, wherein the subscript q is 1.

44. The method of claim 43, wherein L is selected from the group consisting of —CH₂— and —SO₂—.

45. The method of claim 44, wherein the subscript m is 0.

46. The method of claim 39, wherein R^{b} and Ire are each independently selected from the group consisting of C₁-C₈ alkyl, C₁-C₈ alkoxy, phenyl, phenyl (C₁-C₈ alkyl), halogen, —CN, —NH₂, —NH(C₁-C₈ alkyl), —N(C₁-C₈ alkyl)₂, —(C═O)CH₃, —(C═O)NH₂, —OH, —COOH, —COO(C₁-C₈ alkyl), —OCH(C₁-C₈ alkyl), —O(C═O)O(C₁-C₈ alkyl)-NO₂, —SH, —S(C₁-C₈ alkyl), and —NH(C═O)(C₁-C₈ alkyl).

47. The method of claim 39, wherein said compound is selected from the group consisting of:

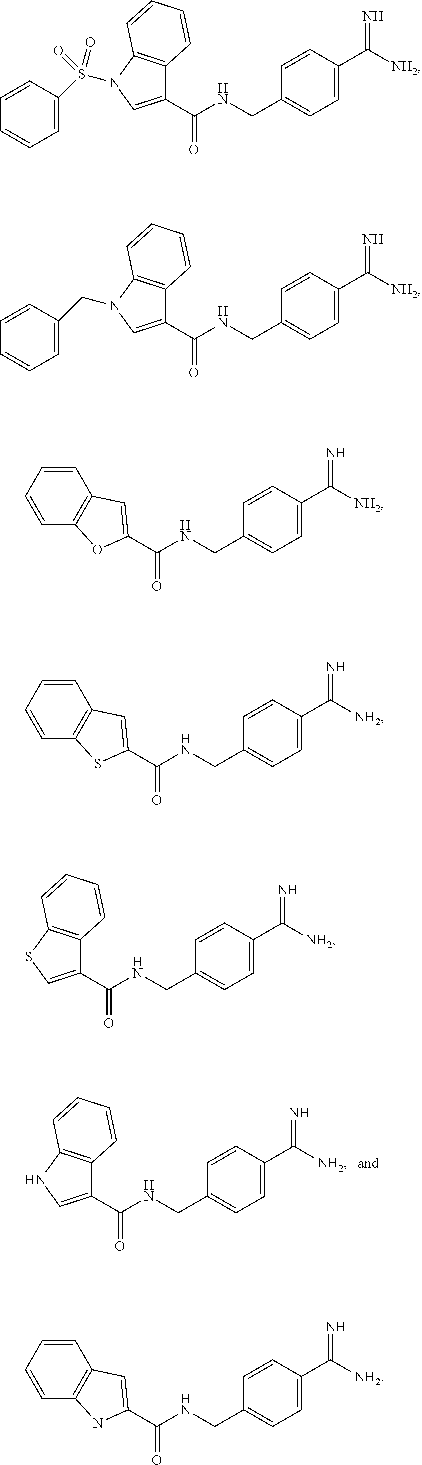

48. The method of claim 1, said method comprising administering to said subject a plasma kallikrein-specific monoclonal antibody.

49. The method of claim 48, wherein the monoclonal antibody is MAB 13G11.

50. The method of any one of claims 1-49, wherein said subject is administered a second agent for reducing blood pressure within one month of said plasma kallikrein inhibitor.

51. The method of claim 50, wherein said second agent is selected from the group consisting of thiazide diuretics, beta blockers, angiotensin-converting enzyme (ACE) inhibitors, angiotensin II receptor blockers, calcium channel blockers, renin inhibitors, alpha blockers, alpha-beta blockers, and vasodilators.

52. A kit comprising:
(a) a plasma kallikrein inhibitor; and
(b) instructions for administering (a) to subject in need of a reduction in blood pressure.

53. The kit of claim 52, wherein said subject suffers from hypertension or prehypertension.
